(19) United States
(12) Patent Application Publication
(10) Pub. No.: US 2008/0236601 A1
(43) Pub. Date: Oct. 2, 2008

(54) MANUFACTURING SHAPE MEMORY POLYMERS BASED ON DEFORMABILITY PEAK OF POLYMER NETWORK

(75) Inventor: Kurt Jacobus, Atlanta, GA (US)

Correspondence Address: GREENBERG TRAURIG, LLP, 1200 SEVENTEENTH STREET, SUITE 2400, DENVER, CO, 80202, US

(73) Assignee: MedShape Solutions, Inc., Atlanta, GA (US)

(21) Appl. No.: 11/692,795

(22) Filed: Mar. 28, 2007

(51) Int. Cl. A61F 2/00

(52) U.S. Cl. 128/899; 264/40.6

(57) ABSTRACT

Methods and systems have been developed allowing for increased strain storage in shape memory polymer devices. Larger stored strains allow for smaller and different implantable shapes of shape memory polymer devices. For example, by storing a larger strain, a shape memory polymer device may be implanted at a smaller fraction of its final implanted shape. Benefits include shorter patient recovery times, smaller implantation sites, and flexibility of implantation techniques.

### BACKGROUND

Shape memory materials are used in medical devices for beneficial results. For example, shape memory materials can be used as a means to reduce the size of surgical entry sites, to reduce recovery time, and to increase bio-compatability of medical devices. Various methods of utilizing shape memory materials have been developed, including methods of producing the materials, methods of manufacturing medical devices with the materials, and methods of surgery using the medical devices, among other methods. Several methods utilize novel materials and novel features of shape memory materials to achieve novel and useful medical and therapeutic results for patients.

Shape memory polymers differ from shape memory alloys and other shape memory materials in the large recoverable strains achievable by shape memory polymers as opposed to other shape memory materials. Medical devices using shape memory materials provide surgeons with the opportunity to perform surgeries with minimally invasive techniques thereby reducing scarring and recovery time. Shape memory materials have the ability to store strains before implantation into a patient and recover an original shape once inside the patient. Because of their ability to store and recover strains, shape memory materials have been used in medical devices with a broadening array of applications.

### SUMMARY

Methods and systems have been developed allowing for increased strain storage in shape memory polymer devices. Larger stored strains allow for smaller and different implantable shapes of shape memory polymer devices. For example, by storing a larger strain, a shape memory polymer device may be implanted at a smaller fraction of its final implanted shape. Benefits include shorter patient recovery times, smaller implantation sites, and flexibility of implantation techniques.

In one aspect the disclosure describes a method including calculating a deformability peak temperature for a shape memory polymer composition, forming a shape memory element from the shape memory polymer composition, wherein the shape memory element is in a first shape, and deforming the shape memory element at about the deformability peak temperature, wherein the deforming is performed from the first shape to a second shape, different from the first shape. The method further includes, after deforming the shape memory element, cooling the shape memory element to a storage temperature below the deformability peak temperature, wherein the deformability peak temperature is below a glass transition temperature of the shape memory polymer composition.

Calculating the deformability peak temperature may include performing a plurality of mechanical strain to failure tests for the shape memory polymer composition. The plurality of mechanical strain to failure tests may include tests selected from tensile tests, compression tests, shear tests, torsion tests, and fracture toughness tests.

Calculating the deformability peak temperature may include calculating a temperature differential between a test transition temperature of a test shape memory polymer composition and a test deformability peak temperature of the test shape memory polymer composition, wherein the test shape memory polymer composition is different from the shape memory polymer composition. Calculating the deformability peak temperature may include subtracting the temperature differential from the test glass transition temperature to calculate the deformability peak temperature.

A part of the shape memory element may retain a tensile engineering strain greater than about 40% between the first shape and the second shape. A part of the shape memory element may retain a compressive engineering strain less than about −50% between the first shape and the second shape.

Activating may include heating the shape memory element to a recovery temperature, wherein the recovery temperature is within 10 degrees Celsius of the transition temperature. Activating may include expanding the shape memory element along an expansion axis into a third shape, wherein the third shape is different from the second shape and different from the first shape. An expansive engineering strain between the second shape and the third shape in a part of the shape memory element may be greater than about 100%.

The method may include creating a medical device comprising a shape memory element, wherein the shape memory element comprises substantially the shape memory polymer composition. The method may include, after cooling the shape memory element, inserting the medical device into a patient, and after inserting the medical device, activating the shape memory element.

In another aspect the disclosure describes a medical device including a shape memory element in a deformed shape, wherein the deformed shape represents an engineering strain along a stretching axis from an unconstrained shape, and wherein the engineering strain is greater than about 100%. The shape memory element may include a shape memory polymer composition including poly-ethylene glycol dimethacrylate in a concentration of about 10% to about 50% by weight, wherein the shape memory element is at a temperature which is less than 10 degrees Celsius below a glass transition temperature of the shape memory polymer composition, and wherein the medical device has a sterile exterior.

The shape memory polymer composition may include a linear chain selected from tert-butyl acrylate and poly-methyl methacrylate. The engineering strain may be greater than about 120%.

### A BRIEF DESCRIPTION OF THE DRAWINGS

FIG. 1A illustrates an example deformation of a shape memory polymer material under compression.

FIG. 1B illustrates an example deformation of a shape memory polymer material under tension.

FIG. 2A shows a modulus curve and strain to failure data for a ten percent by weight crosslinker shape memory polymer composition.

FIG. 2B shows two graphs of strain to failure tests using the same shape memory polymer composition tested in FIG. 2A.

FIG. 2C shows a modulus curve and strain to failure data for a twenty percent by weight crosslinker shape memory polymer composition.

FIG. 2D shows a modulus curve and strain to failure data for a forty percent by weight crosslinker shape memory polymer composition.

FIG. 2E shows the strain to failure data for three shape memory polymer compositions (e.g., 10%, 20% and 40% weight by crosslinker compositions).

FIG. 3 shows a flowchart of a method for utilizing a shape memory polymer in a medical device.

### DETAILED DESCRIPTION

The following description of various embodiments is merely exemplary in nature. While various embodiments have been described for purposes of this specification, various changes and modifications may be made to the embodiments disclosed herein.

Shape memory materials are materials which are able to be deformed from a memorized or unconstrained shape into a different shape and have that different shape remain stable for a period of time before the shape memory material is activated and returns toward its unconstrained shape. The process of creating a memorized shape can include deforming a shape memory material into a deformed shape and cooling it to a storage temperature. Cooling to a storage temperature serves to keep the shape memory material in the deformed shape and avoid activation of the shape memory material.

Activation may include subjecting the shape memory material to a stimulus such as heat, radiation, chemicals or other stimuli known to those with skill in the art. Activation is discussed further below.

The shape memory material may not activate to exactly its unconstrained shape depending on the physical constraints of the shape memory material's surroundings while it is activated. Therefore, activation may result in a shape memory material reaching its unconstrained shape, but that does not need to be the case. In some instances, activation may result in very little shape change. As used herein, the term unconstrained shape will refer to a shape of a shape memory material after activation without any significant constraint. The shape of a shape memory material after the material has been deformed may be referred to as a deformed shape, a stored shape, an implantation shape, or simply a second shape.

A glass transition temperature of a shape memory polymer (SMP) may be calculated using several methods. For example, a glass transition temperature may be calculated from a change in modulus of the SMP, from a peak tan-delta measurement, or from other measurements known to those with skill in the art. However, the term glass transition temperature, as used in this disclosure and in the claims is the temperature at which the inflection point occurs on the modulus-temperature curve of the SMP such as illustrated herein.

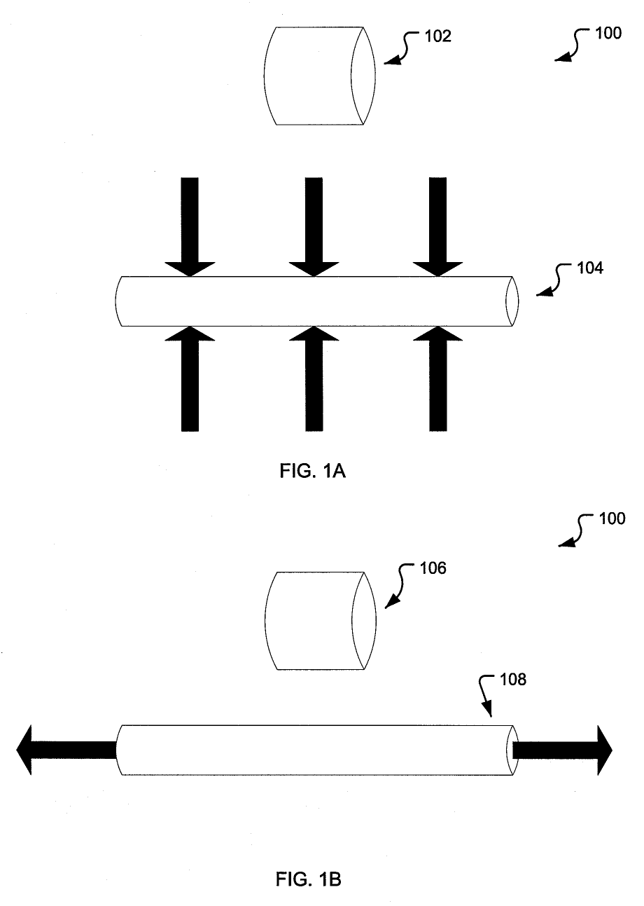

FIG. 1A illustrates an example deformation of a shape memory polymer material under compression. FIG. 1A shows the original shape (above) and the deformed shape (below). The original shape above is compressed along the vertical axis (e.g., the compressing axis) into the deformed shape, as denoted by the arrows. The shape elongates along the horizontal axis in response to the compression along the vertical axis.

FIG. 1B illustrates an example deformation of a shape memory polymer material under tension, or via stretching. FIG. 1B shows the original shape (above) and the deformed shape (below). The original shape above is stretched under tension along the horizontal axis (e.g., the elongating axis) into the deformed shape, as denoted by the arrows. The shape contracts along the vertical axis in response to the tension along the horizontal axis.

FIGS. 1A and 1B show two different methods of achieving similar deformed shapes. While results from tension and compression may not be exactly or precisely analogous, as described further below, there are useful analogies which may be drawn between the two methods of deformation. For example, a temperature at which a peak strain under tension is achievable may be used to calculate a temperature at which a peak strain under compression may be approached.

When strain is discussed herein using numerical values, it should be understood as engineering strain. Engineering strain is known by those with skill in the art. For clarity, engineering strain is defined along an axis. The axis used to define engineering strain herein is the axis along which deformation forces are being applied. For example, when a deformation is caused by compression, the axis along which the compression is provided, or the compressing axis (in FIG. 1A, the vertical axis), is the axis along which engineering strain is measured. Alternatively, when a deformation is caused by tension, the axis along which the tension is provided, or the elongating axis (e.g., in FIG. 1B, the horizontal axis), is the axis along which engineering strain is measured. Deformations as a result of compressions produce negative engineering strains because the difference of length along the compressing axis is a negative difference. Thus, when negative strains are referred to herein, a greater strain actually represents a smaller deformation (e.g., negative 50% is greater than negative 80%), and a lesser strain represents a larger deformation. Deformations which are a result of tension produce positive engineering strains because the difference in length along the tension axis is a positive difference.

Some examples of deformations are illustrated with respect to FIGS. 1A and 1B. The deformations illustrated therein are simple tension and compression deformations with the deformation evenly and equally applied along one direction of the shape memory element (e.g., radial, longitudinal). These types of deformations can apply substantially consistent strains to several parts of the shape memory element. For example, in FIG. 1A, the compressing force along the vertical axis (or, in another embodiment, along a plurality of radial axes) induces a compressing strain along this axis or axes that is substantially consistent across parts of the shape memory element. As another example, the strain illustrated in FIG. 1B also demonstrates a global strain which is likely to be substantially reflected in consistent local strains. In this example, both the global strains and local strains are tensile strains.

In other words, parts of the shape memory element may each store a strain which is substantially consistent with the global strain imposed on the entire shape memory element. As an example, if the entire shape memory element is subjected to a global compression strain of −50%, each part of the shape memory element may have a substantially consistent local strain. Put another way, if the entire shape memory element is compressed to a −50% compression strain, many parts of the shape memory element may be compressed to a −50% compression strain.

In contrast to the strains in the deformations illustrated in FIGS. 1A and 1B, there are virtually limitless types of deformations possible with a shape memory element. The strain to failure tests illustrated in FIGS. 1A and 1B may be used to measure (on a global basis) failure strains which may occur only on a local basis in more complex deformation configurations. In other words, in some instances, the tested deformation along a particular axis for the entire shape memory element will be represented by most, if not all, of the parts of the shape memory element storing a similar strain.

As an example of the number and type of deformations possible apart from the illustrated deformations, deformation may be performed through various combinations of twisting, folding, extruding, shearing, bending, or torquing. Such deformations may be achieved on a shape memory element using varying degrees of stress across the shape memory element and using various combinations of forces.

In addition to the number and variety of deformations which may be performed on a shape memory element on a global basis, there are at least an equal number of various deformations which may occur on a local level to parts of the shape memory element. For example, when the shape memory element is globally bent, some parts of the shape memory element will be locally under compression and some parts will be under tension. In addition, interface regions may be under a shear form of deformation between parts under compression and parts under tension.

As another example, if a shape memory element is formed in the shape of a lattice and that lattice is deformed globally in one manner (e.g., through compressing or crushing the lattice), then parts of the lattice may be deformed locally in another manner, such as through being bent, compressed, or stretched. The engineering strains measured on a global basis for a shape memory element may vary markedly in type and magnitude from the strains stored on a local basis in various parts of the shape memory element. For example, a shape memory element which globally is compressed to an engineering strain of −50% may have parts of the shape memory element which have local strains which are relatively greater such as compression strains of −75% or less or tension strains of 100% or more.

A large number of possible global deformations of a shape memory element embody a complex multitude of local strains distributed through the various parts of the shape memory element. Often the local strains will not be consistent among each other nor will they match the global strain of the shape memory element, and thus failure strains may be approached locally by one or more parts of the shape memory element before a global strain nears the failure strain. For example, whereas a shape memory element may embody a global strain which is well below the failure strain, there may be parts of the shape memory element which embody strains which are very near or past the failure strain. In other words, part of the shape memory element may be strained to failure before other parts of the shape memory element are strained to failure. Thus, when strains are described herein, they may refer to local strains in parts of shape memory elements, even if that strain is located only in one part or region of the shape memory element. Local strains can be predicted from global deformations of strains through theoretical, experimental, analytical or computational methods known to those skilled in the art.

Suitable shape memory polymers described herein have polymer compositions of tert-butyl acrylate (tBA) as a linear chain and poly-ethlyene glycol (PEG) as a crosslinker. Other linear chains, such as poly-methyl methacrylate (PMMA) could be used with similar results. Linear chains may be chosen based on properties of SMP materials. For example, ranges of achievable rubbery moduli may vary between compositions of SMPs based on the linear chain used in the composition. Some compositions may use poly-ethlyene glycol dimethacrylate (PEGDMA) as a crosslinker. The SMP compositions shown herein, which may be characterized as thermosets or thermoplastics, as appropriate, were composed to have substantially consistent glass transition temperatures. A photo-initiator suitable for initiating polymerization between the linear chain and crosslinker was used in sufficient weight quantities to polymerize the composition. The compositions were polymerized using ultraviolet light.

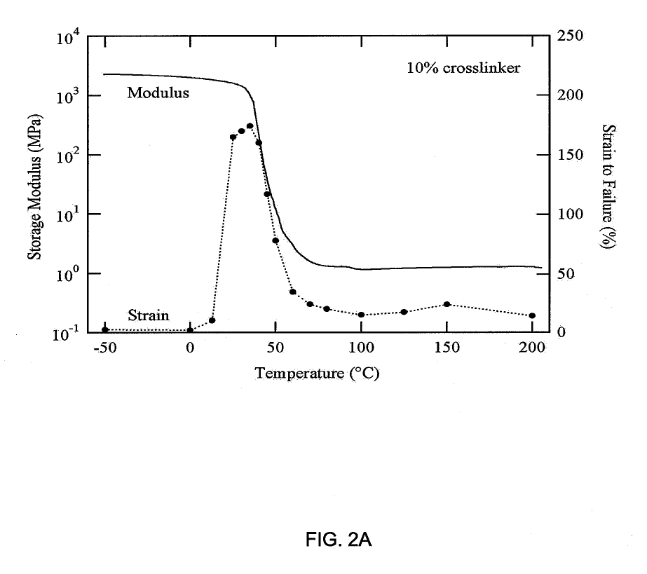

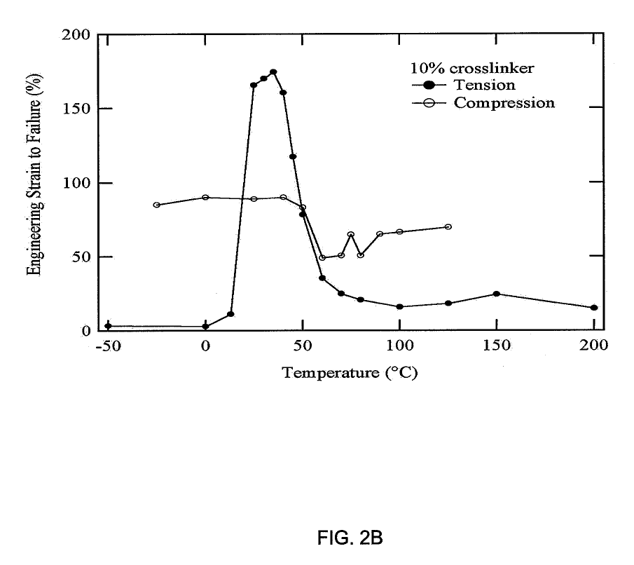

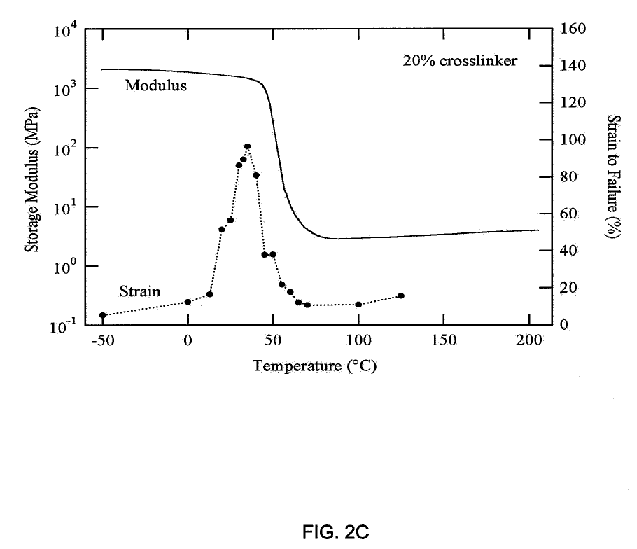

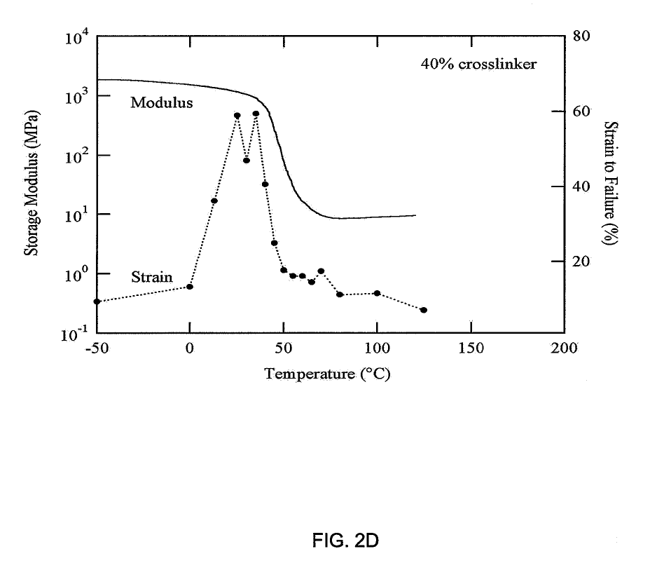

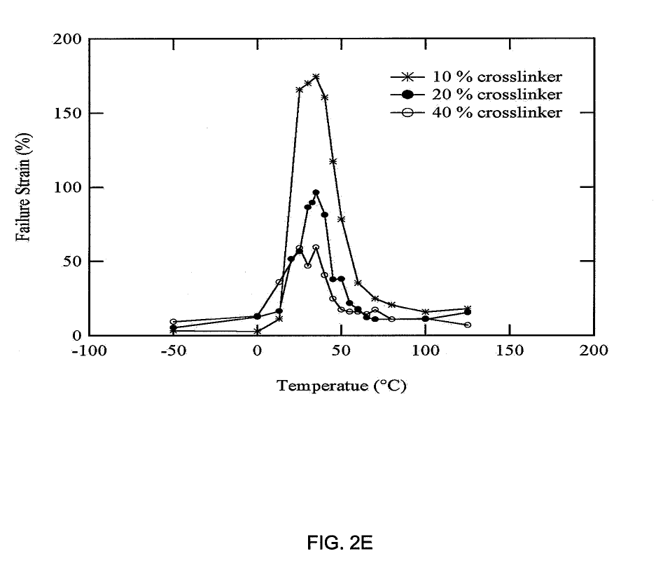

FIGS. 2A-2E show test results of shape memory polymers of various compositions. Each of the compositions in FIGS. 2A-2E is a composition of tBA-PEGDMA mixed at the noted weight percentage of crosslinker (PEGDMA). Even though results shown in FIGS. 2A-2E disclose compositions with 10%-40% by weight crosslinker, this disclosure is applicable to compositions with percentages by weight of crosslinker in excess of 40%. For example, experiments have been performed at 45% and greater percentage weights of crosslinker. Other percentage weights of crosslinker may be selected for different applications and/or to select a rubbery modulus for the SMP composition. As described further below, FIG. 2E compares the properties of the various SMP compositions to each other.

FIG. 2A shows a modulus curve and strain to failure data for a ten percent by weight crosslinker shape memory polymer composition. Samples of SMP were created and tested using a dynamic modulus analysis (DMA) machine to test for transitions in the modulus at different temperatures. As described above, there are different methods of calculating a glass transition temperature for a SMP.

Also in FIG. 2A are data taken from strain to failure tests. In the strain to failure tests, SMPs of the same SMP composition were created and strained under tension to failure (e.g., stretched under tension to a point of failure). The samples were stretched under tension at various temperatures and the failure strain (i.e. the strain at which the SMP failed) was recorded. For example, for the composition of FIG. 2A at 200° Celsius, the failure strain or the strain achievable before failure of the SMP composition was roughly 10-15%. At 50° Celsius, the failure strain under tension was roughly 75% strain.

In each of FIGS. 2A-E, several SMP samples were used to determine the temperatures at which the failure strain peaked and became smaller. A series of strain to failure tests is one way to calculate deformability peak of a SMP. Any of the deformations described herein may form the basis of strain to failure tests which may be used to calculate deformation peak temperatures from the failure strains observed at different temperatures. Indeed, many types of mechanical tests may be used to calculate deformation peak temperatures via strain to failure tests. Other tests which may be used to calculate deformability peak temperatures include fracture toughness tests, or tests which measure the resistance to the extension of a crack within a body while the body is strained. Other ways of calculating the deformability peak of a SMP are discussed below.

The term deformability peak should not be construed to refer only to one exact temperature, but rather the term is better defined as a range of temperatures covering an increase and subsequent decrease in deformability. The term deformability itself should be understood as a property related to the amount of failure strain attainable by a SMP (e.g., ability to undergo strain without failure). As shown in FIG. 2A, the deformability peak may be a range of temperatures at which the SMP exhibits a high degree of deformability.

In FIG. 2A, there are four samples which each demonstrated the ability to undergo greater than 150% deformation before failing. Each of these temperatures may be considered a deformability peak temperature or a temperature within the deformability peak of the SMP because each of these temperatures produced a failure strain near a “peak” failure strain. In one embodiment, a deformability peak temperature may be considered any temperature which produces a failure strain within 20% of this peak failure strain. This peak failure strain is indicated by the failure strains rising and then subsequently falling for different SMP samples held at different temperatures.

Deformability peak temperatures may include temperatures below the glass transition temperature where the failure strain increases beyond the failure strains recorded around or above the glass transition temperature. For example, in FIG. 2A, the four failure strain data points above 150% strain are each greater (in this example, markedly) than the failure strain near the glass transition temperature. In FIG. 2A, each of the four failure strain data points have nearly 50% greater strain than the failure strain near the glass transition temperature.

It may be impractical to find a specific temperature which produces an absolute peak of failure strain, and an approximation of the peak may be sufficient to create a SMP with a high degree of stored strain. For example, the four samples demonstrating failure strains of greater than 150% show a variation of failure strains between the four samples of about 20% to 25% and any of these failure strains may be sufficiently large for a given application. Therefore, the entire temperature range covered by the temperatures may also be used as a deformability peak temperature. A range of deformability peak temperatures may be useful to give flexibility to other manufacturing constraints when creating devices with SMPs.

FIG. 2B shows two graphs of strain to failure tests using the same SMP composition tested in FIG. 2A. FIG. 2B shows compression data in relation to tension data. The solid circles represent the same data points shown in FIG. 2A of the strain to failure tests under tension of the SMP samples. The open circles show data of strain to failure tests under compression of samples of the same SMP composition as in FIG. 2A. As an illustration, the compression tests shown by the open circles may be visualized as a similar test to the compression shown in FIG. 1A. It should be noted that the engineering strain referenced in the compression data in FIG. 2B is actually negative, but is graphed on the graph in FIG. 2B as a positive number for comparison purposes.

Also of note in FIG. 2B is that the compression data plateaus at a temperature of around 50° Celsius. This plateau is a result of the fact that compression engineering strain reaches a theoretical maximum at 100%. This is because of the definition of engineering strain as a difference in length divided by an original length. In other words, at the theoretical maximum of 100% compression engineering strain, the new length along the compression axis is zero (0) length, i.e., non-existent.

In addition, the plateau of the compression data is partially a result of the testing module used for the compression tests which had a maximum compression force of 30 kilonewtons (kN). After the sharp increase in failure strain of the compression test, the machine compressing the SMP material reached its maximum compressive force and so no further engineering strain was able to be recorded.

Regardless of these real world and theoretical limitations, the compression data shows a correlation with the tension data. Thus, a deformability peak temperature derived under tension may also be used when compression is the desired form of deformation. In addition, compression data may be used when tension is the desired form of deformation.

FIG. 2C shows a modulus curve and strain to failure data for a twenty percent by weight crosslinker shape memory polymer composition. A modulus curve shows the transition temperature is similar to the transition temperature of the 10% by weight crosslinker SMP composition. Strain to failure tests were also performed on this composition and the results of those tests are denoted by the solid circles. A deformability peak may be calculated for this SMP composition from these samples in a similar manner as described in FIG. 2A.

As described in relation to FIG. 2A, in FIG. 2C there are several points around the deformability peak which may all be considered as part of the deformability peak temperature. For example, the four points of strain to failure above 80% could all be considered to be part of a deformation peak. Particularly, the three points that are above 90% might be considered to be part of a deformability peak. Each of the temperatures at which these peak failure strain results were recorded may be considered a deformability peak temperature for this SMP composition.

FIG. 2D shows a modulus curve and strain to failure data for a forty percent by weight crosslinker shape memory polymer composition. The strain to failure data was obtained in a similar manner to those described in FIG. 2A and FIG. 2C. As in those previous Figures, in FIG. 2D the strain to failure data shows a range of temperatures which may be considered the deformability peak temperature. For example, deformability peak temperatures for the SMP composition in FIG. 2D may be considered temperatures corresponding to the three points showing a failure strain over 40%. As discussed above, each one of these deformability peak temperatures is below the transition temperature shown on the modulus curve.

FIG. 2E shows the strain to failure data for three shape memory polymer compositions (e.g., 10%, 20% and 40% weight by crosslinker compositions). Each of these compositions has a different range of peak failure strains due to the different amounts of crosslinker in each of the compositions. For example, in the data shown, the SMP composition with 10% weight by crosslinker has the highest failure strain data at over 150%. The differences between the SMP compositions may be understood as reflecting the differences in the polymer chemistry between the polymer compositions.

It should be noted that the glass transition temperatures and the deformability peak temperatures for each of the SMP compositions did not significantly change between the SMP compositions used for FIGS. 2A-2E. Furthermore, the glass transition temperatures of the compositions did not change significantly, even though the percentage weight of crosslinker ranged from 10% to 40%.

As demonstrated in FIGS. 2A-2E, different SMP compositions may exhibit a consistent relationship between glass transition temperature and deformability peak temperatures. For example, FIG. 2E shows that relationship is a consistent differential between the temperatures because the deformability peaks are substantially consistent, and, as shown in FIGS. 2A, 2C, and 2D, the glass transition temperatures are substantially consistent between the SMP compositions. A consistent differential between glass transition temperature and deformability peak temperature for one SMP composition may be used as an estimate of the differential between the glass transition temperature and deformability peak temperature for another SMP composition.

In each of the SMP compositions shown in FIGS. 2A-2E, the deformability peak temperature is below the transition temperature of the SMP composition, so the differential may be used as an estimate of how much lower the deformability peak temperatures are below the glass transition temperature. For example, a differential of about 10 degrees Celsius may be determined in one set of tests on one SMP composition, and the same differential of about 10 degrees Celsius may be used to estimate a deformability peak temperature for another SMP composition by subtracting the 10 degree differential from the glass transition temperature of the other SMP composition.

Established procedures for utilizing shape memory properties of shape memory materials including SMPs are commonly taught in the art. A technique described in the art includes deforming a SMP at a temperature above the glass transition temperature, holding the SMP in the deformed shape, and then cooling the SMP while in the deformed shape to a storage temperature which is below the transition temperature. This technique results in the SMP substantially holding the deformed shape while the SMP is at the storage temperature. In this procedure, the SMP is activated by heating the SMP.

Many teachings in the art teach that strains (e.g., deformations) created below the glass transition temperature may not be recoverable. Other teachings presume that because activation (change to the memorized or unconstrained shape) significantly occurs above the glass transition temperature that deformation should also occur above the glass transition temperature.

As described herein, and in contrast to the prior art, deformation below the glass transition temperature of a SMP can allow greater strains to be stored by the SMP. As shown in FIGS. 2A-2E, the achievable strains for SMPs above the transition temperature may be much smaller than the achievable strains for the SMP at its deformability peak. One benefit of a larger stored strain is the SMP may be inserted into a patient while the SMP is in a smaller form. Medical devices with SMPs containing greater strains may utilize smaller insertion sites into a patient, may decrease recovery times, and provide surgeons with greater choice of surgical techniques, among other benefits.

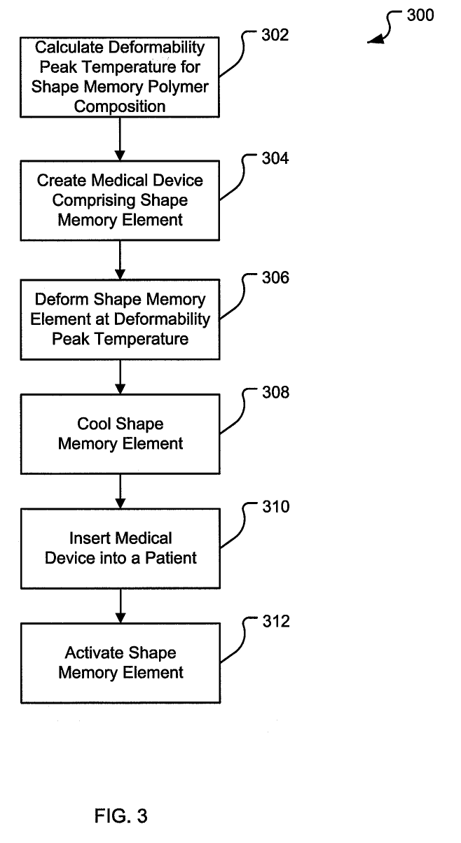

FIG. 3 shows a flowchart of a method 300 for utilizing a shape memory polymer in a medical device. In the embodiment shown, the method includes calculating a deformability peak temperature 302 for a SMP composition. Calculating a deformability peak temperature may be performed using any of the tests or methods described above.

In addition, calculating a deformability peak temperature 302 for a SMP composition may be performed using a look-up table comprising data which is recorded from tests performed by others. In one embodiment, calculating a deformability peak temperature 302 may be performed using data may be stored in scientific literature known to those with skill in the art and calculating a deformability peak temperature for a particular SMP composition may be performed by reviewing the literature and making extrapolations therefrom. In another embodiment, calculating a deformability peak temperature 302 may be performed using the data in FIGS. 2A-2E. For example, the temperature difference between the transition temperature and the deformability peak temperature of the SMPs tested therein may be calculated and used to estimate a deformability peak temperature for other SMP compositions.

In one embodiment, calculating a deformability peak temperature 302 may be performed via calculating a difference between the glass transition temperature and a deformability peak temperature for one SMP composition and using that temperature difference as an estimate for the same difference of temperatures in another SMP composition. This type of estimating may be used even if the SMP compositions are significantly different. For example, the estimate of deformability peak temperature may be used in initial tests and later refined for use with a different SMP composition.

Using such an estimate would also comprise an embodiment of calculating the deformability peak temperature for the SMP composition of interest.

In one embodiment, the method includes creating a medical device 304 which includes a shape memory element. The shape memory element may comprise the entire medical device or the shape memory element may comprise a part of the medical device. For example, a SMP may be shaped into the form of a medical device, such as a graft fixation device. As illustrated in FIGS. 1A-1B, a medical device may be created in a shape resembling the cylindrical shape of a wine cork. As another example, a shape memory element may be used as a shape changing element in the medical device which has other elements which do not change shape, such as rigid anchors, supporting structures, or the like.

In another embodiment, the method does not include making a medical device comprising the shape memory element. For example, the shape memory element may be a SMP which is manufactured separately from the medical device and included in the medical device in a later process or by another entity. As another example, a shape memory element comprising a SMP may be used in another application different from a medical device for use with a patent.

In one embodiment, the method includes deforming the shape memory element 306 at the deformability peak temperature. Deforming a shape memory element 306 may be performed in any of the manners described above including tension, compression or a combination thereof or another form of deformation.

The deformability peak temperature at which the deformation is performed 306 may be a precise temperature or one of a range of temperatures, as described above. There are several methods known to those with skill in the art of assuring that the shape memory element is at a temperature during deformation. One such method of insuring the temperature is to place the shape memory element in a temperature controlled environment. Another such method is to place the shape memory element in contact with a heating/cooling element and to monitor the shape memory element's temperature as the deformation takes place.

There are other methods of controlling the temperature of a shape memory element while the deformation is performed and these methods would readily suggest themselves to those with skill in the art. For example, different methods of controlling the temperature of a shape memory element may be more suitable than others depending on how the deformation is performed. As described above, the deformability peak temperature may be a range of temperatures. Based on how wide a range of temperatures is considered the deformability peak temperature, the temperature at which the shape memory element is held during deformation may be exactly controlled or alternatively controlled within a few degrees Celsius.

In the embodiment shown, the method includes cooling the shape memory element 308 after the deformation is performed. The cooling of the shape memory element 308 is performed in order to bring the shape memory element to a storage temperature as defined above which minimizes activation to an acceptable amount. In other words, at the deformation peak temperature, the shape memory element may recover some of the strain imparted during the deformation process if the shape memory element is held at the deformability peak temperature. This is because, as described above, activation or recovery of strain toward the unconstrained shape may occur at temperatures near the glass transition temperature even if the glass transition temperature is not attained. Therefore, cooling the shape memory element 308 below the deformability peak temperature aids in retaining the strain imparted to the shape memory element during the deformation process.

After the shape memory element is cooled 308 to a storage temperature, it may be packaged, shipped and/or stored at the storage temperature. As described further herein, the storage temperature may vary somewhat around the temperature to which the shape memory element was cooled after deformation. Such a variation in temperature may be expected as the shape memory element is transferred from the manufacturing site to a site where the shape memory element is used. However, so long as the shape memory element is not allowed to recover significant strains, these temperature variations should not affect the usefulness of the shape memory element.

One embodiment of the method includes inserting the medical device 310 containing the shape memory element into a patient. The inserting 310 may be performed as described in the art and known to those with skill in the art. However, as described above, the medical device may be smaller due to the large strains stored by the shape memory element. For example, as illustrated in FIG. 2A, the shape memory element deformed above the glass transition temperature for the SMP may only be able to store an engineering strain of 25% before failing. However, a shape memory element as described herein may be able to store a strain of 175% engineering strain. Therefore, the shape memory element as described herein may have a much different form factor than a shape memory element made using conventional techniques.

In one embodiment, the method includes activating the shape memory element 312. Activating the shape memory element 312 may be performed as described herein including through heating the shape memory element to bring it above a transition temperature (e.g., a glass transition temperature for a SMP), through electromagnetic radiation, through chemical activation, and through other techniques. In one embodiment, the method does not include inserting a medical device into a patient or activating the shape memory element. As described above, the method may include the processes of manufacturing the shape memory element and another entity may perform the integration of the shape memory element into a medical device inserting the medical device into the patient and activating the shape memory element inside the medical device.

The term activation should not be construed to be a SMP changing shape to its original or unconstrained shape. Rather, as used herein, activation may result in varying degrees of actual shape change, depending on constraints placed on the SMP. Activation, therefore, may result in a post-activation shape which may be different than an unconstrained shape.

For a heat-activated SMP, activation may begin to occur at an onset temperature. As known by those with skill in the art, the onset temperature is below the glass transition temperature. In other words, the glass transition temperature need not be attained for the SMP to be activated. As is known in the art, some activation in a heat-activated SMP (e.g., release of the stored strain) can occur below the transition temperature. For example, activation will begin at a temperature which is below the glass transition temperature, but is above the onset temperature.

A storage temperature may be chosen such that, at or below the storage temperature, significant activation is unlikely to occur. In other words, a storage temperature may be chosen below the onset temperature to inhibit significant activation.

The speed of activation may be affected by the temperature of the SMP and its relation to the glass transition temperature. For example, activation may be achieved below the glass transition temperature at a slower rate than above the glass transition temperature. Thus, the speed of activation may be affected by the temperature of the SMP and its relation to the glass transition temperature.

Portions of these embodiments may be performed separately from each other as well as in combination with each other. Portions of the embodiments may be performed at different times or at times separated by breaks or pauses. For example, the calculating may be separated from the deforming which may be separated from the inserting.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

What is claimed is:

1. A method comprising:
calculating a deformability peak temperature for a shape memory polymer composition;
forming a shape memory element from the shape memory polymer composition, wherein the shape memory element is in a first shape;
deforming the shape memory element at about the deformability peak temperature, wherein the deforming is performed from the first shape to a second shape, different from the first shape;
after deforming the shape memory element, cooling the shape memory element to a storage temperature below the deformability peak temperature; and
wherein the deformability peak temperature is below a glass transition temperature of the shape memory polymer composition.

2. The method of claim 1, wherein calculating the deformability peak temperature comprises:
performing a plurality of mechanical strain to failure tests for the shape memory polymer composition.

3. The method of claim 2, wherein the plurality of mechanical strain to failure tests include tests selected from tensile tests, compression tests, shear tests, torsion tests, and fracture toughness tests.

4. The method of claim 2, wherein the plurality of mechanical strain to failure tests are tensile strain to failure tests.

5. The method of claim 1, wherein calculating the deformability peak temperature comprises:
calculating a temperature differential between a test glass transition temperature of a test shape memory polymer composition and a test deformability peak temperature of the test shape memory polymer composition, wherein the test shape memory polymer composition is different from the shape memory polymer composition; and
subtracting the temperature differential from the test glass transition temperature to calculate the deformability peak temperature.

6. The method of claim 1, wherein a part of the shape memory element retains a tensile engineering strain greater than about 40% between the first shape and the second shape.

7. A shape memory element created by the process of claim 6.

8. The method of claim 6, wherein the part of the shape memory element retains a tensile engineering strain greater than about 80% between the first shape and the second shape.

9. The method of claim 8, wherein the part of the shape memory element retains a tensile engineering strain greater than about 150% between the first shape and the second shape.

10. The method of claim 1, wherein a part of the shape memory element retains a compressive engineering strain less than about −50% between the first shape and the second shape.

11. The method of claim 10, wherein the part of the shape memory element retains a compressive engineering strain less than about −80% between the first shape and the second shape.

12. The method of claim 1, wherein activating comprises:
heating the shape memory element to a recovery temperature, wherein the recovery temperature is within 10 degrees Celsius of the glass transition temperature.

13. The method of claim 1, wherein activating comprises:
expanding the shape memory element along an expansion axis into a third shape;
wherein the third shape is different from the second shape and different from the first shape.

14. The method of claim 13, wherein an expansive engineering strain between the second shape and the third shape in a part of the shape memory element is greater than about 100%.

15. The method of claim 1, wherein the method further comprises:
creating a medical device comprising a shape memory element, wherein the shape memory element comprises substantially the shape memory polymer composition.

16. The method of claim 15, wherein the method further comprises:
after cooling the shape memory element, inserting the medical device into a patient; and
after inserting the medical device, activating the shape memory element.

17. A medical device comprising:
a shape memory element in a deformed shape, wherein the deformed shape represents an engineering strain along a stretching axis from an unconstrained shape;
wherein the engineering strain is greater than about 100%;
wherein the shape memory element comprises a shape memory polymer composition including poly-ethylene glycol dimethacrylate in a concentration of about 10% to about 50% by weight;
wherein the shape memory element is at a temperature which is less than 10 degrees Celsius below a glass transition temperature of the shape memory polymer composition; and
wherein the medical device has a sterile exterior.

18. The medical device of claim 17, wherein the shape memory polymer composition further comprises:
a linear chain selected from tert-butyl acrylate and poly-methyl methacrylate.

19. The medical device of claim 17, wherein the engineering strain is greater than about 120%.

20. The medical device of claim 19, wherein the engineering strain is greater than about 150%.
